(19) United States
(12) Patent Application Publication
(10) Pub. No.: US 2017/0290969 A1
(43) Pub. Date: Oct. 12, 2017

(54) METHOD AND APPARATUS FOR PREDICTING ONE OR MORE PARAMETERS CHARACTERISTIC FOR THE OUTCOME OF A BLOOD TREATMENT

(71) Applicant: FRESENIUS MEDICAL CARE DEUTSCHLAND GMBH, Bad Homburg (DE)

(72) Inventors: Maria MILLAN-GALANTE, Bad Homburg (DE); Erik DUELSNER, Florstadt (DE); Wolfgang WEHMEYER, Tuebingen (DE)

(73) Assignee: FRESENIUS MEDICAL CARE DEUTSCHLAND GMBH, Bad Homburg (DE)

(21) Appl. No.: 15/512,742

(22) PCT Filed: Sep. 15, 2015
(86) PCT No.: PCT/EP2015/001846

§ 371 Date: Mar. 20, 2017

(30) Foreign Application Priority Data
Sep. 18, 2014 (DE) 10 2014 013 886.0

(51) Int. Cl. A61M 1/16; A61M 1/34; B01D 61/14; A61B 5/00

(52) U.S. Cl. CPC A61M 1/1613; A61B 5/4875; A61M 1/34; B01D 61/145

(57) ABSTRACT

The present invention relates to a method and to an apparatus for predicting one or more parameters characteristic for the outcome of a blood treatment, wherein the blood treatment is a treatment in which the blood of the patient has fluid removed via at least one membrane, wherein the parameters are the allowed drinking volume, the hyperhydration or hypohydration of the patient, the clearance of large molecules and/or the allowed salt intake, wherein the prediction of the allowed drinking volume and the prediction of the hyperhydration or hypohydration are carried out on the basis of the planned weight loss due to ultrafiltration, of the drinking quantity during the treatment, of the rinseback volume and of residual diuresis data, and/or wherein the prediction of the clearance of large molecules is carried out on the basis of the urea clearance, and/or wherein the prediction of the allowed salt intake takes place based on the sodium ion quantity removed by ultrafiltration and by diffusion from the blood.

The present invention relates to a method and to an apparatus for predicting one or more parameters characteristic for the outcome of a blood treatment, wherein the blood treatment is a treatment in which the blood of the patient has fluid removed via at least one membrane.

A major aim in dialysis in addition to the removal of substances from the blood is the removal of water from the patient's blood. Patients requiring dialysis cannot eliminate water taken in with food again or can only eliminate again insufficiently. This water collects in the patient's blood and in the patient's tissue from where it can again flow into the patient's vascular system.

This excess water is removed from the patient's blood by the ultrafiltration which takes place within the framework of the dialysis treatment, i.e. by the water removal from the blood via the membrane of the dialyzer. The ultrafiltration volume is frequently taken as a measure as to which volume of fluid the dialysis patient can take in during two dialysis treatments (allowed drinking volume).

If the ultrafiltration volume is used as a measure for the allowed drinking volume, there is a disadvantage in that this is in this respect not an exact indication for the allowed drinking volume.

It is the underlying object of the present invention to further develop a method and an apparatus of the initially named kind such that a prediction or calculation of parameters which are characteristic for the outcome of a blood treatment is carried out which is as exact as possible.

This object is achieved by a method having the features of claim 1 as well as by an apparatus having the features of claim 13. Provision is accordingly made that the parameters in question are the allowed drinking volume and/or the hyperhydration or hypohydration of the patient and/or the clearance of large molecules and/or the allowed salt intake.

If the parameter to be predicted is the allowed drinking volume up to the next treatment or also the hyperhydration or hypohydration which results after the current or upcoming treatment, these parameters are predicted in accordance with the invention on the basis of the planned weight loss of the patient due to ultrafiltration, of the drinking volume during the treatment, of the rinseback volume and of the volume removed by the residual diuresis.

If the parameter to be predicted is the clearance of large molecules, in particular the clearance of microglobolin, provision is made in accordance with the invention that the prediction of this clearance is carried out on the basis of the urea clearance.

If the parameter to be predicted is the allowed salt intake up to the next dialysis treatment, the prediction of this parameter takes place on the basis of the sodium ion quantity removed by ultrafiltration and by diffusion from the blood.

It is possible by the method in accordance with the invention and by the apparatus in accordance with the invention to deliver to the patient or to the user of a blood treatment device, in particular a dialysis device, an indication which is as accurate as possible on the value of one or more parameters which is adopted in dependence on values which play a role within the framework of the blood treatment or after its termination or will play a role at the end of the treatment.

It is thus possible, for example, to deliver a comparatively accurate prediction of the allowed drinking volume up to the next treatment on the basis of the named values using the method in accordance with the invention or using the apparatus in accordance with the invention.

It is thus possible to determine the consequence of a programming of a blood treatment device, for example by inputting the ultrafiltration quantity, etc., and to display the result to the patient or to the user of the device in a suitable manner.

The advantages result from this that the user or the operator can still intervene during the treatment or also before the treatment when the predicted parameters do not adopt desired values or lie outside desired value ranges.

The patients or users can read off the consequences directly after they have intervened in or carried out the programming of the blood treatment device and can optionally correct the settings again.

A further advantage comprises the patient receiving higher self-responsibility and an amplification of awareness of the disease or of the therapy by outputting the allowed drinking volume up to the next blood treatment. Any conflicts between the patient and nursing staff are also remedied in that an objective parameter value is displayed by the method or by the apparatus.

If the predicted parameter is the allowed drinking volume, it can be calculated from the planned weight loss due to ultrafiltration less the drinking volume during the treatment and less the rinseback volume and plus the volume removed by residual diuresis and less or plus a volume which is led in or let out or is not led in or let out due to interventions during the ongoing treatment, as is, for example, the case with a reduced ultrafiltration rate.

The rinseback volume is a part of a fluid volume of substituate or saline solution with which the blood is pressed out of the extracorporeal blood circuit back into the vascular system of the patient at the end of the treatment. A small part of the substituate or of the saline solution in this respect also moves into the vascular system or is also mixed with a small part of the patient's blood. This part is the rinseback volume. This can be estimated, for example, or can also be stored as a piece of information in the machine control.

The volume produced by residual diuresis is the volume of fluid which is removed from the patient's blood between two blood treatments due to the residual renal activity.

The permitted drinking quantity up to the next blood treatment or up to the next dialysis treatment is calculated from the net balance of weight loss or fluid removal and weight gain or fluid intake or reduced fluid removal.

A further important dialysis parameter is the hyperhydration or hypohydration of the patient. It is determined from the difference of the predicted weight of the patient after the treatment and the normal weight, i.e. the normohydration of the patient. If both values correspond to one another, there is thus neither a hyperhydration nor a hypohydration.

The predicted weight of the patient after the treatment is determined from his predialysis weight less the planned weight loss due to ultrafiltration plus a drinking volume during the treatment and plus the rinseback volume. The volume removed by residual diuresis is optionally also deducted. The volume is furthermore taken into account which is led in or led off due to interventions during the ongoing treatment.

The interventions or factors which have an influence on the volume or on the fluid balance during the ongoing treatment can, for example, be a bolus administration and/or a change in the treatment duration and/or a change in the ultrafiltration target. An example for such an effect is, for example, the shorter time, i.e. the shortening of the treatment duration which results in a smaller removed fluid volume with an unchanged ultrafiltration rate.

The rinseback volume and/or the volume gained by residual diuresis can be taken from a memory or from a database, for example, or can also be estimated. In general, one or both of these parameters can e.g. be stored as a piece of information in the machine control.

It is conceivable that the actual weight variation or the weight of the patient and/or the volume removed due to ultrafiltration or the ultrafiltration rate and/or the drinking volume during the treatment are detected by suitable measuring devices.

It is thus possible that the patient's weight or the weight change is monitored by a set of scales over time. The monitoring of the ultrafiltration quantity or ultrafiltration rate by one or more flow sensors or the like is equally possible. The drinking volume during the treatment can be made known to the blood treatment device via any desired user interface (for example, a smart phone, a remote control or the like). A monitoring of the patient by a camera and the transfer of the information with respect to the drinking volume acquired in this manner to the dialysis device is also conceivable.

A further informative parameter is the average hyperhydration TAFO (time average fluid overload). Treatment data of past blood treatments are made use of to present this value. The value of the average hyperhydration is preferably formed as an average value from the values of the hyperhydration before and after preceding treatments as well as from the value of the hyperhydration before the current treatment and from the predicted value of the hyperhydration after the current treatment.

The clearance of large molecules and in particular of microglobulin (β2M) can also be estimated by the present method. In this respect, the urea clearance is used as the basis and an estimate of the clearance of large volumes is carried out on the basis of this value. Provision is preferably made that the urea clearance is determined by determining or measuring the conductivity in the consumed dialyzate. Such a method is known, for example, from EP 1444997 B1 to which reference is made in this respect. The distribution volume required for the estimate of the urea clearance can be determined by measuring the bioimpedance of body parts and of body components derived therefrom such as fat, water and muscle mass.

A further important parameter is the estimate of the permitted salt intake up to the next dialysis treatment or blood treatment.

Salt is removed from the patient during the blood treatment or dialysis by diffusion and by convection or by ultrafiltration via the membrane of the dialyzer. The patient's intake should not substantially exceed this quantity of salt up to the next treatment. To determine the removed salt quantity, the current plasma concentration of sodium in the blood plasma can be determined. Such a method is known from WO 2010/112223 A1 to which reference is made in this respect. The sodium quantity which is removed from the blood by means of ultrafiltration via the membrane can then be estimated using this value of the plasma concentration. Furthermore, the quantity of diffuse substance transport of sodium ions can be estimated if the value over the total body water and the difference of predialytic and postdialytic plasma concentrations are considered.

Also like the further parameters, the permitted salt intake up to the next dialysis can also be output in a suitable manner so that the recommendation of the physician is correspondingly backed up and the patient is given an objective value on which he can orientate himself.

As stated above, the predicted parameter(s) and/or one or more of the values which influence the parameters in question are output by means of at least one output apparatus. In this respect it is preferably a monitor and in particular a touchscreen monitor. Other output apparatus are also generally conceivable and covered by the invention.

Provision is made in a further embodiment of the invention that a change of the predicted value of the parameter is calculated and is output at the output apparatus. If the user or the patient thus, for example, carries out a change of a value which has an influence on the parameter to be predicted such as a change of the ultrafiltration volume, provision can be made that the consequence resulting from this is calculated and is output directly to the patient or to the user. It is thus possible, for example, on a change of the ultrafiltration volume, to display directly to the patient what consequences this change can have for the allowed drinking volume or also for the allowed salt intake up to the next treatment.

There is preferably the possibility that one or more values can be input by a user which have an influence on the predicted value of the parameter.

It is thus possible that the physician or the patient reacts directly to the prediction by an amended prescription.

The present invention furthermore relates to an apparatus for predicting one or more parameters characteristic for the outcome of a blood treatment in accordance with the features of claim 13.

Further preferred embodiments of the apparatus are the subject of dependent claims 14 to 24.

The present invention furthermore relates to a blood treatment apparatus, in particular to a dialysis device, for carrying out a blood treatment having an extracorporeal circuit in which a filter or dialyzer is arranged. It is flowed through by blood on one side and, depending on the type of treatment, by dialyzate that flows through a dialyzate circuit on the other side. Individual substances from the blood pass via the membrane preferably configured as a hollow fiber bundle and a volume reduction or water removal of the patient's blood takes place by ultrafiltration.

The blood treatment device can comprise the apparatus in accordance with claims 13 to 24 or can have at least one such apparatus.

Further details and advantages of the invention will be explained in more detail with reference to an embodiment explained in the drawing. There are shown:

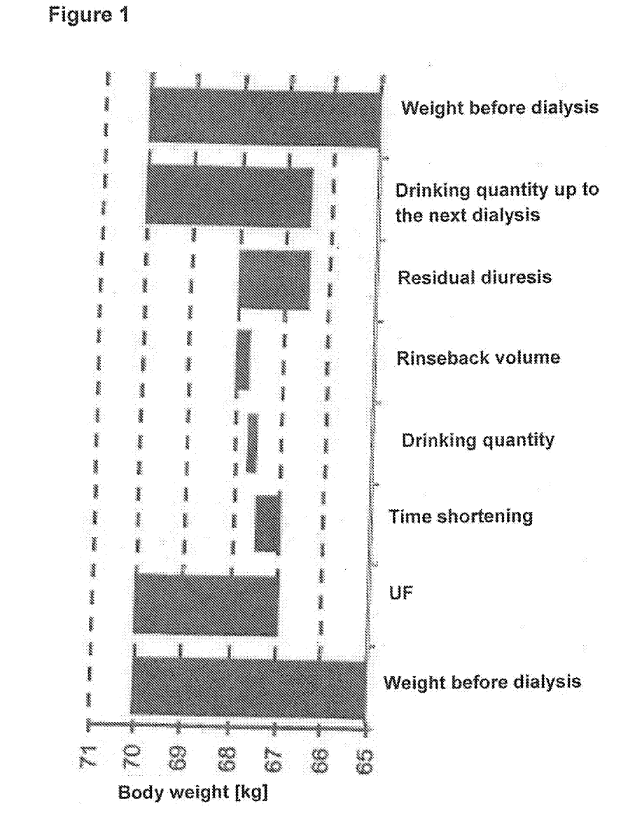

FIG. 1: a representation of the body weight in dependence on different influence variables for determining the allowed drinking volume up to the next treatment; and

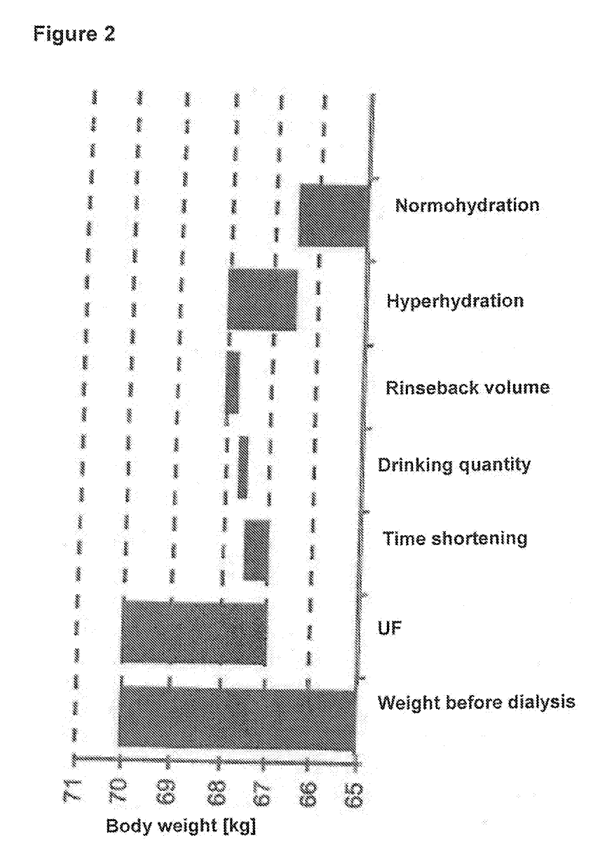

FIG. 2: a representation of the body weight in dependence on different values for determining the hyperhydration of the patient after the treatment.

In FIG. 1, the body weight of a dialysis patient is shown as well as different values which have an influence on the body weight after dialysis. The allowed drinking quantity up to the next dialysis treatment is likewise shown.

In FIG. 1, the patient's weight before the dialysis is shown in the right and left bar, with the lower end of the bar being cut-off for reasons of clarity. As can be seen from FIG. 1, the patient has a body weight of 70 kg before the dialysis. A certain quantity of fluid should be removed from the patient by ultrafiltration during the dialysis to achieve a so-called dry weight. In the present example, the ultrafiltration quantity is set to 3 kg, corresponding to 3 liters fluid removal. This results from the UF bar in accordance with FIG. 1.

The behavior of the patient and any changed parameters during the dialysis can, however, change the net fluid removal so that the allowed drinking volume does not necessarily have to correspond to the removed ultrafiltration volume. As can be seen from FIG. 1, in addition to the planned weight loss in the current treatment (data from the dialysis machine), consequences of the interventions made in the ongoing treatment, e.g. on the ultrafiltration quantity, play a role. These consequences or interventions are summarized in FIG. 1 under the term “time shortening”. In addition to a time change, a bolus administration or also a change of the ultrafiltration goal can be covered by this. In the example shown here, the time shortening is a parameter which results in an increase in the volume or in the body weight at the end of the treatment since the time shortening has the consequence of a reduction in the ultrafiltration volume with an unchanged ultrafiltration rate.

The “drinking quantity” bar illustrates the volume of fluid the patient takes in during the ongoing treatment or will take in within the framework of the upcoming treatment. Another source of fluid is the volume of substituate or saline solution which is marked as the rinseback volume in the Figure.

While the three values “time shortening”, “drinking volume” and “rinseback volume” in the example shown here result in an increase in the body weight or body volume, the residual diuresis, i.e. the residual renal activity, results in a reduction in body weight and body volume up to the next treatment. The allowed drinking quantity up to the next dialysis, which is shown as the 2nd bar from the right in FIG. 1, is calculated from the net balance of weight loss through fluid removal and weight increase by fluid intake or reduced fluid removal over the duration of the dialysis.

As can be seen from FIG. 1, the bars of the events (UF and residual diuresis) which result in a fluid removal from the patient extend downward. The bars of the events (time shortening, drinking quantity and rinseback volume) which result in a fluid increase of the patient extend upward. As can be seen from FIG. 1, the individual events are shown next to one another. In this respect, the base lines from which the bars respectively extend (with the exception of the right and left bars which represent the weight before dialysis) are formed by the end point of the respective left neighboring bar. The bar “UF” thus starts at the end point of the bar for the weight of the patient, i.e. at the line at 70 kg representing the weight of the patient, and extends downward starting from this because ultrafiltration is a fluid removal. The extent by which the UF bar extends downward corresponds to the fluid quantity removed within the course of the ultrafiltration. A corresponding procedure applies to the further events. The bar for the time shortening extends starting from the lower end point of the UF bar. This bar extends upward because the time shortening results in a fluid increase or in less removed fluid. The bar of the drinking volume adjoins the end point of the bar of time shortening, etc.

The allowed drinking quantity up to the next dialysis treatment then simply results from the difference of the end point of the first or last bar (dry weight of the patient) and the end point of the last bar (residual diuresis).

A corresponding procedure applies to the representation in accordance with FIG. 2.

It is generally also possible and covered by the invention to allow the bars of the events which result in a fluid removal from the patient to extend upward and to allow the bars of the events which result in a fluid increase of the patient to extend downward.

The drinking volume which the patient takes in during the current treatment can be provided in the form of data via the dialysis machine or by a database. This applies accordingly to the rinseback volume and also to the residual diuresis. These data can, for example, be determined and provided via the patient's card or also via a network from a patient database.

FIG. 2 shows a representation of the body weight in dependence on different values for determining the parameter “hyperhydration”. Hyperhydration represents an important piece of information for the physician at the end of the ongoing dialysis, before the next dialysis or also as a mean hyperhydration value over two or more dialysis treatments.

As can be seen from FIG. 2, the hyperhydration of the patient is determined at the end of the ongoing dialysis from the weight before the dialysis (data of the scales) or from the patient's card or via a network less the normohydration weight. This can also be obtained from the patient's card or via a network, for example.

If a net fluid removal takes place in the course of the treatment to a degree such that the normohydration weight is reached at the end of the treatment, no hyperhydration is present.

In the embodiment shown here, the ultrafiltration rate, the time shortening and other special effects, the drinking volume and the rinseback volume play a role for the calculation how much body fluid is removed. Apart from the residual diuresis, the same values are thus used as a basis as in the embodiment in accordance with FIG. 1 so that reference is made accordingly.

The hyperhydration which is shown as the second bar from the right in FIG. 2 results from the weight before the dialysis and from the probable fluid removal less the normohydration. A hypohydration of the patient can accordingly also result at the end of the dialysis depending on the removed fluid quantity.

The hyperhydration or hypohydration can be shown as an absolute value, e.g. in liters, or also after division by the normohydration weight as a relative hyperhydration or hypohydration in percent. As a rule, the hyperhydration relates to the value relative to the normohydration and is generally not zero after the prescription of the physician (dry weight, weight after dialysis). Positive values or negative values are possible. The hyperhydration or hypohydration is essentially determined by the cardiovascular stability of the patient toward the end of the dialysis.

A further informative parameter which can be output in accordance with the invention is the average hyperhydration TAFO. This value helps the physician to estimate the average cardiovascular strain on the body by hyperhydration. It is conceivable to carry out the determination of the hyperhydration over a 7-day interval, i.e. as a rule over the last three dialysis treatments.

In detail, the result is in this case

TAFO=⅙*[(Ü1pre+ÜW2pre+ÜW3pre)+(ÜW1post+ÜW2post+ÜW3post)]

Here, the values ÜW1pre, ÜW2pre represent the hyperhydration before the dialysis from preceding dialysis treatments 1 and 2 and the values ÜW1post and ÜW2post represent the hyperhydration values after the dialysis of these preceding treatments 1 and 2. The data can be obtained from database information, e.g. from the patient's card, via a network or from an internal memory of the dialysis machine.

In the example shown here, the value ÜW3pre relates to the magnitude of the hyperhydration which results from the weight before the current dialysis, obtained, for example, by a measurement by scales, less the normohydration. The value ÜWpost is the prediction of the hyperhydration for the current treatment which can be calculated or predicted as described above. The effective actual value of ÜW3post can be stored in a database after the treatment to be available for the following calculations of the average hyperhydration.

The measurement of the clearance of large molecules such as microglobolin is determined in accordance with the invention partly by measured values and partly by stored values. The starting point for the estimate of the average clearance of large molecules is an estimate of the filter performance for urea. The technical relationship between the urea clearance and the clearance of large molecules is stored in performance maps and is therefore known. The relative performance of the clearance of large molecules for urea clearance can first be estimated from this.

Known methods can be used for calculating the urea clearance such as the measurement of the conductivity in the consumed dialyzate. Reference is made to EP 1 444 997 B1 in this respect. In this respect, the measurement of the clearance or of the dialysance of urea is carried out at the dialyzate side.

Further parameters which can enter into this calculation can be flow parameters of the current treatment such as the blood flow, the dialyzate flow, the ultrafiltration rate and, in hemodiafiltration or hemofiltration, the infusion flow. Further parameters are the treatment mode (hemodialysis, hemodiafiltration and hemofiltration) and the product data of the blood filter or dialyzer.

As soon as the urea clearance has been determined, a “calibration” to the actual operating conditions can take place. A prediction on the average clearance of urea up to the end of the dialysis can be made from the programmed data for the flows. The prediction of the infusion volumes is also considered here. Based on the average clearance of urea, a forecast or a conclusion can then be drawn by means of calibration curves or performance maps on an average clearance of large molecules, in particular of microglobolin. This value can then also be displayed accordingly and it is possible to influence the clearance by changes of certain parameters such as by changes of flow rates and likewise to have the changed value displayed.

Errors in the calculation can arise in that the relationship of urea clearance and the clearance of large molecules is variable due to the special composition of the blood of a patient. To alleviate or remedy this problem, the following correction parameters are conceivable:

Concentration of clotting factors, hematocrit, albumin and protein in the blood (data from the blood analysis of the patient);

increase in the concentration of clotting factors, hematocrit, albumin and protein during the dialysis by thickening of the blood as a result of the ultrafiltration (data from the blood volume sensor during the treatment).

The presentation of the result, i.e. the prediction of the average clearance of large molecules can take place, for example, as an average clearance or also as a performance value in the form of the product from clearance and time as well as also as a relative performance value in the form of the product of clearance and time divided by the distribution volume of the large molecules. This can be determined as shown above by the measurement of the bioimpedance of body parts and of body components derived therefrom such as fat, water and muscle mass.

It is possible by the present invention to display to the physician or to the user of the device which value parameters essential to the treatment may adopt after the treatment. This prediction makes it possible to intervene immediately if unwanted values are obtained.

In addition to a presentation on a screen, any other desired representation options or output options are also conceivable such as an acoustic voice output or a printout.

It has previously been assumed for both the drinking volume and the salt intake that the patient status in the next dialysis should be the same as in the current blood treatment. There is, however, the possibility that the physician would like to make a slight correction with respect to the hyperhydration. According to the current prior art, this correction in the next dialysis is pursued by a reduction of the target weight, which brings about a higher ultrafiltration quantity and thus a larger strain on the body.

In accordance with the present invention, this correction, for example a volume loss of 200 ml, can already be set by the physician before the current dialysis and can be transmitted to the dialysis device in a suitable manner by input means. This correction can then enter into the representation of the permitted drinking quantity, namely in the form of the calculated drinking quantity less the correction. In this case, the calculated drinking quantity less 200 ml results as the permitted drinking volume. The same also applies accordingly to the salt intake.

1. A method for predicting one or more parameters characteristic for the outcome of a blood treatment, wherein the blood treatment is a treatment in which the blood of the patient has fluid removed via at least one membrane, characterized in that the parameters are the allowed drinking volume, the hyperhydration or hypohydration of the patient, the clearance of large molecules and/or the allowed salt intake, wherein the prediction of the allowed drinking volume and the prediction of the hyperhydration or hypohydration are carried out on the basis of the planned weight loss due to ultrafiltration, of the drinking quantity during the treatment, of the rinseback volume and of residual diuresis data, and/or wherein the prediction of the clearance of large molecules is carried out on the basis of the urea clearance, and/or wherein the prediction of the allowed salt intake takes place based on the sodium ion quantity removed by ultrafiltration and by diffusion from the blood.

2. A method in accordance with claim 1, characterized in that the predicted allowed drinking volume is calculated from the planned weight loss due to ultrafiltration less the drinking volume during the treatment less the rinseback volume plus the volume removed by residual diuresis and less or plus volume which is led in or led off due to interventions during the ongoing treatment.

3. A method in accordance with claim 1, characterized in that the predicted hyperhydration or hypohydration at the end of the treatment is calculated from the difference of the predicted weight of the patient after the treatment and the normal weight (normohydration) of the patient, wherein the predicted weight of the patient after the treatment is calculated from the predialysis weight less the planned weight loss due to ultrafiltration plus the drinking volume during the treatment plus the rinseback volume less the volume removed by residual diuresis and less or plus volume which is led in or led off due to interventions during the ongoing treatment.

4. A method in accordance with claim 2, characterized in that the interventions are a bolus administration, a change of the treatment duration and/or a change of the ultrafiltration target.

5. A method in accordance with claim 1, characterized in that the rinseback volume and/or the volume gained by residual diuresis is obtained from a memory.

6. A method in accordance with claim 1, characterized in that the weight of the patient and/or the volume removed by ultrafiltration and/or the drinking volume is measured during the treatment.

7. A method in accordance with claim 1, characterized in that the prediction of the hyperhydration is the prediction of the average hyperhydration (TAFO) which also covers values of the hyperhydration of preceding treatments.

8. A method in accordance with claim 7, characterized in that the average hyperhydration is calculated by the relationship
TAFO=1/n* [(ÜW1, pre+ÜW(n−1),pre+ÜWn,pre)+(ÜW1,post +ÜW(n−1),post+ÜWn,post)]
where ÜW1,pre and ÜW(n−1),pre and ÜW1,post and ÜW(n−1)post are values of the hyperhydration before and after preceding treatments 1 . . . (n−1) respectively, wherein ÜWn,pre is the value of the hyperhydration before the current treatment determined from the difference of the patient's weight and the normohydration, and where ÜWn,post is the predicted value of the hyperhydration after the current treatment.

9. A method in accordance with claim 1, characterized in that the determination of the urea clearance takes place by measurement of the urea concentration in the dialyzate or by measurement of the conductivity of the dialyzate.

10. A method in accordance with claim 1, characterized in that the value of the predicted parameter(s) and/or one or more of the values influencing it is output by means of at least one output apparatus, with the output apparatus preferably being a monitor and in particular being a touchscreen monitor.

11. A method in accordance with claim 10, characterized in that a change in the predicted value of the parameter is calculated and is output at the output apparatus.

12. A method in accordance with claim 1, characterized in that one or more values of variables can be input by a user which have an influence on the predicted value of the parameter.

13. An apparatus for predicting one or more parameters characteristic for the outcome of a blood treatment, wherein the blood treatment is a treatment in which the blood of the patient has fluid removed via at least one membrane, characterized in that the parameters are the allowed drinking volume, the hyperhydration or hypohydration of the patient, the clearance of large molecules and/or the allowed salt intake, wherein the apparatus has calculation means which are configured such that they carry out the prediction of the allowed drinking volume and the prediction of the hyperhydration or hypohydration on the basis of the planned weight loss due to ultrafiltration, of the drinking quantity during the treatment, of the rinseback volume and of residual diuresis data, and/or such that they carry out the prediction of the clearance of large molecules on the basis of the urea clearance, and/or that they carry out the prediction of the allowed salt intake based on the sodium ion quantity removed by ultrafiltration and by diffusion from the blood.

14. An apparatus in accordance with claim 13, characterized in that the calculation means are configured such that the predicted allowed drinking volume is calculated from the planned weight loss due to ultrafiltration less the drinking volume during the treatment less the rinseback volume plus the volume removed by residual diuresis and less or plus volume which is led in or led off due to interventions during the ongoing treatment.

15. An apparatus in accordance with claim 13, characterized in that the calculation means are configured such that the predicted hyperhydration or hypohydration at the end of the treatment is calculated from the difference of the predicted weight of the patient after the treatment and the normal weight (normohydration) of the patient, wherein the predicted weight of the patient after the treatment is calculated from the predialysis weight less the planned weight loss due to ultrafiltration plus the drinking volume during the treatment plus the rinseback volume less the volume removed by residual diuresis and less or plus volume which is led in or led off due to interventions during the ongoing treatment.

16. An apparatus in accordance with claim 14, characterized in that the interventions are a bolus administration, a change of the treatment duration and/or a change of the ultrafiltration target.

17. An apparatus in accordance with claim 13, characterized in that the apparatus has at least one memory in which the rinseback volume and/or the volume gained by residual diuresis is stored.

18. An apparatus in accordance with claim 13, characterized in that the apparatus has at least one measuring device for measuring the weight of the patient and/or the volume removed by ultrafiltration and/or of the drinking volume during the treatment.

19. An apparatus in accordance with claim 13, characterized in that the calculation means are configured such that they calculate the value of the average hyperhydration (TAFO), with values of the hyperhydration of preceding treatments also entering into the calculation.

20. An apparatus in accordance with claim 19, characterized in that the calculation means are configured such that the average hyperhydration is calculated in accordance with the relationship
TAFO=1/n*[(ÜW1,pre+ÜW(n−1),pre+ÜWn,pre)+(ÜW1,post+ÜW(n−1),post+ÜWn,post)]
where ÜW1,pre and ÜW(n−1),pre and ÜW1,post and ÜW(n−1)post are values of the hyperhydration before and after preceding treatments 1 . . . (n−1) respectively, wherein ÜWn,pre is the value of the hyperhydration before the current treatment determined from the difference of the patient's weight and the normohydration, and where ÜWn,post is the predicted value of the hyperhydration after the current treatment.

21. An apparatus in accordance with claim 13, characterized in that the calculation means are configured such that the determination of the urea clearance takes place on the basis of one or more measured values of the urea concentration in the dialyzate or of the conductivity of the dialyzate.

22. An apparatus in accordance with claim 13, characterized in that the apparatus has at least one output apparatus which is configured such that the value of the predicted parameter(s) and/or one or more of the values influencing it is output by means of the output apparatus, with the output apparatus preferably being a monitor and in particular being a touchscreen monitor.

23. An apparatus in accordance with claim 22, characterized in that the calculation means are configured such that they determine a change in the predicted value of the parameter; and in that the output apparatus is configured such that the determined value is output at the output apparatus.

24. An apparatus in accordance with claim 13, characterized in that the apparatus has one or more input means which are configured such that one or more values of variables can be input by a user which have an influence on the predicted value of the parameter.

25. A blood treatment apparatus, in particular a dialysis device, for carrying out a blood treatment, in which the blood of the patient has fluid removed via at least one membrane, characterized in that the blood treatment apparatus has at least one apparatus in accordance with claim 13 or is formed by at least one apparatus in accordance with claim 13.
